(19) United States
(12) Patent Application Publication
(10) Pub. No.: US 2007/0104789 A1
(43) Pub. Date: May 10, 2007

(54) Gastro-resistant and ethanol-resistant controlled-release formulations comprising hydromorphone

(76) Inventor: Donald Spector, New York, NY (US)

Correspondence Address: DIEHL SERVILLA LLC, 77 BRANT AVE, SUITE 110, CLARK, NJ, 07066, US

(21) Appl. No.: 11/266,918

(22) Filed: Nov. 4, 2005

(51) Int. Cl. A61K 9/24; A61K 31/485

(52) U.S. Cl. 424/472; 514/282

(57) ABSTRACT

The invention provides a controlled-release composition comprising hydromorphone or a pharmaceutically acceptable form thereof in association with a gastro-resistant and ethanol-resistant compound and method of use thereof.

### BACKGROUND OF THE INVENTION

Hydromorphone is a potent opioid used for chronic pain relief. Until recently, hydromorphone was available on the market as Palladone™, which is a controlled-release capsule formulation. In this formulation, hydromorphone is released into a patient's bloodstream gradually, thus allowing the patient to take the hydromorphone capsule only once per day. Gradual release of hydromorphone is important because hydromorphone in high doses has significant and dangerous side effects. Among these effects are respiratory depression, hypotension, carbon dioxide retention, and elevation of cerebrospinal fluid pressure, which may lead to death of the patient. Thus, it is important that hydromorphone be released into the patient's bloodstream gradually, rather than acutely.

Recently, it has been found that alcohol increases release of hydromorphone from Palladone™, leading to adverse effects so severe that FDA has recently asked the manufacturer of Palladone™ to voluntarily remove Palladone™ from the market. (See Jul. 13, 2005 FDA Alert For Healthcare Professionals. www.fda.gov/cder/drug/InfoSheets/HCP/hydromorphoneHCP.htm. According to the FDA release, concentrations of hydromorphone in the blood increased over fivefold when 12 mg Palladone™ capsules (the lowest available dose) were taken with 8 ounces of 40% (80 proof) alcohol. Even smaller amounts of alcohol consumed by certain patients accelerated release of hydromorphone. For example, studies showed that taking Palladone™ with a mixed drink (equivalent to 20% alcohol) or even with one beer (equivalent to 4% alcohol) result in increased hydromorphone concentration, compared to taking Palladone™ without alcohol.(Id.) Accordingly, there is an immediate need for a gastro-resistant and ethanol-resistant controlled release hydromorphone formulation.

### SUMMARY OF THE INVENTION

The present invention fills the foregoing need by providing such a gastro-resistant and ethanol-resistant controlled release hydromorphone formulation. In one aspect, the invention provides a controlled-release composition comprising hydromorphone or a pharmaceutically acceptable form thereof in association with a gastro-resistant and ethanol-resistant compound. The composition may comprise hydromorphone in a matrix of a gastro-resistant and ethanol-resistant compound. Alternatively, the gastro-resistant and ethanol-resistant compound may form a coating around the matrix containing hydromorphone. Further, the gastro-resistant and ethanol-resistant compound may be present in both the matrix and the coating of the composition. The composition may be formulated as a capsule, a tablet, a pill, a pellet or a granule.

In another aspect, the invention provides a method of treating a mammal, including humans having chronic pain comprising orally administering to such mammal the controlled-release composition described above once a day.

### DETAILED DESCRIPTION OF THE INVENTION

For the purposes of this application, the following definitions shall apply:

The term “hydromorphone” shall mean hydromorphone or any pharmaceutically acceptable salt thereof, including without limitation hydromorphone hydrochloride.

The term “gastro-resistant” shall mean the ability to delay or prevent the release of hydromorphone in the acidic gastric medium of a patient's stomach so that the hydromorphone is released gradually into the patient's bloodstream without any adverse effects. The term “ethanol-resistant” shall mean the ability to delay or prevent the release of hydromorphone induced by alcohol consumption.

Generally, the embodiments of the present invention relate to a composition comprising hydromorphone in association with a gastro-resistant and ethanol-resistant compound. The composition may be formulated as a capsule, a tablet, a pill, a pellet, or a granule.

Examples of suitable gastro-resistant and ethanol-resistant substances include, but are not limited to alginic acid, glyceryl monostearate, glyceryl palmitostearate, carnauba wax, microcrystalline wax, white wax, yellow wax, and ethylcellulose with less than 47% of ethonyl groups, as described in Handbook of Pharmaceutical Excipients, Fourth Edition, (edited by Rowe, Sheckey, and Weller, Pharmaceutical Press, 2003). The formulations according to embodiments of the present invention may comprise auxiliary excipients such as for example diluents, binders, lubricants, surfactants, disintegrants, plasticisers, anti-tack agents, opacifying agents, pigments, and the like. As will be appreciated by the people skilled in the art, the exact choice of excipient and their relative amounts will depend to some extent on the final oral dosage form. Suitable diluents include, for example, pharmaceutically acceptable inert fillers such as microcrystalline cellulose, lactose, starch, dibasic calcium phosphate, saccharides, and/or mixtures of the foregoing. Examples of microcrystalline celluloses include (Avicel PH 200, Avicel PH 102, Avicel PH 112, Avicel PH 101, Avicel PH 3020. Non-limiting examples of lactose include lactose monohydrate. In addition, mannitol, sucrose, and dextrose may be used. Suitable binders include, for example, starch, povidone, low viscosity hydroxypropylmethylcellulose such as Methocel E-5 Prem. LV, pregelatinised starch, hydroxypropylcellulose and/or mixtures of the foregoing. Suitable disintegrants include, for example, crosslinked polyvinyl pyrrolidone, various starches, such as potato starch, corn starch, rice starch, and modified starches, crospovidone, sodium starch glycollate croscarmellose sodium, and the like or mixtures thereof. Suitable lubricants, including agents that act on the flowability of the powder to be compressed are, for example, colloidal slilcon dioxide such as Aerosil® 200; talc; stearic acid, magnesium stearate, calcium stearate and sodium stearyl fumarate.

As indicated above, the dosage forms of the present invention may comprise auxiliary, excipients such as for example lubricants, plasticisers, anti-tack agents, opacifying agents,) pigments, and such like. As will be appreciated by those skilled in the art, the exact choice of excipients and their relative amounts will depend to some extent on the final oral dosage form.

A person skilled in the art will recognize that the gastro-resistant, and ethanol-resistant substances can be used in the matrix, as well as the coating of the composition. Accordingly, in one embodiment, the present invention is a composition comprising hydromorphone and a gastro-resistant and ethanol-resistant substance, wherein the gastro-resistant and ethanol-resistant substance serves as a matrix of the composition.

Non-limiting examples of the gastro-resistant and ethanol-resistant substances especially suitable for the matrix of the composition include glyceryl monostearate and glyceryl palmitostearate.

The composition according to embodiments of the present invention may be prepared by many methods known to a skilled artisan. For example, granules for preparing tablets according to the invention can be manufactured in accordance with standard procedures in which hydromorphone may be combined with suitable excipients prior to mixing and forming compressible granules by adding solution of a binder in a low or high shear mixer or by fluidized bed granulation. The granules are dried, preferably in a fluidized bed dryer. The dried granules are sieved and mixed with lubricants and disintegrants. Alternatively, the manufacture of granules can be achieved by direct mixing of the directly compressible excipients or by roller compaction.

In some cases, it may be desirable to provide a formulation, wherein a patient receives a small predetermined dose of hydromorphone immediately upon ingestion of the formulation, while the bulk of hydromorphone is released slowly over time. Thus, it would be desirable in certain embodiments to select a coating which includes the predetermined amount of the hydromorphone and an encapsulating agent which is readily dissolved in either the gastric fluid of a patient or alcohol. Non-limiting examples of such agents are chitosan, poly(butyl methacrylate, (2-dimethyl aminoethyl) methacrylate, methyl methacrylate) 1:2:1 (also known as Eudragit E 100, produced by Röhm GmBH), and poly(ethyl acrylate, methyl methacrylate) 2:1 (also known as Eudragit E 12.5, produced by Röhm GmBH).

After ingestion of the formulation, the encapsulating agent would dissolve quickly, thus releasing said predetermined amount of hydromorphone. At the same time, the bulk of hydromorphone in complex with the gastro-resistant and ethanol-resistant compound will not be immediately available to the patient and will be released slowly upon time. A person skilled in the art will recognize that different embodiments are possible to achieve this goal. For example, a composition with more than one coating is possible. In this embodiment, the outermost coating will quickly disintegrate in the gastric environment thus releasing hydromorphone, while the bulk of hydromorphone can be protected by an inner coating comprising the gastro-resistant and ethanol-resistant compound.

In another embodiment, the present invention comprises a composition including hydromorphone and one or more gastro-resistant and ethanol-resistant substances, serving as coating agents for the formulation. Non-limiting examples of gastro-resistant and ethanol-resistant substances suitable for coating are carnauba wax, microcrystalline wax, white wax, yellow wax, and ethyl cellulose with less than 46.5% of ethonyl groups. It is preferred that amount of gastro-resistant and ethanol-resistant coating applied is from 5% to 30% by weight with regard to the total weight of the matrix of the composition.

The first step of the process for the preparation of the composition with controlled release of hydromorphone is anhydrous granulation of hydromorphone and dried pharmaceutically acceptable auxiliary substances so that their weight loss at drying is preferably less than 1.0%. Organic solvents to be used in the process of anhydrous granulation should, preferably, contain less than 0.2% of water. The process of anhydrous granulation is carried out in such a way that a dried surfactant is dissolved in an organic solvent at room temperature and the obtained solution is sprayed in a fluidized bed granulator onto a homogenous powdery mixture containing hydromorphone, a dried binder soluble in organic solvents, a dried cellulose ether and other dried pharmaceutically acceptable auxiliary substances. The organic solvents to be used for that purpose are selected from the group of alcohols, ketones, esters, ethers, aliphatic hydrocarbons, halogenated hydrocarbons, cycloaliphatic, aromatic, heterocyclic solvents and mixtures thereof. The plastic mixture obtained in the process of anhydrous granulation is formed into granules or pellet cores by common pharmaceutical technological processes such as extruding and spheronizing methods. The pellet cores or granules so formed are dried in a fluidized bed or in a chamber dryer at the temperature of inlet air from 35° C. to 45° C. until the weight loss at drying is less than 1.0% of the total weight of the pellet cores or granules. Under the addition of dried pharmaceutically acceptable auxiliary substances, dry pellet cores or granules may be compressed into tablets, which in further procedure are coated with a gastro-resistant, ethanol resistant coating. In view of the porosity of the tablets, also one or more intermediate coatings may be applied between the tablet and the gastro-resistant and ethanol-resistant coating. Alternatively, pellet cores or granules prepared by means of anhydrous granulation may be coated with gastro-resistant and ethanol-resistant coating and then filled into capsules or bags or compressed into tablets under addition of dried pharmaceutically acceptable auxiliary substances. In view of the porosity of pellet cores or granules, also one or more intermediate coatings may be applied between the pellet core or granule and the gastro-resistant coating.

The dosage forms of the present invention may comprise auxiliary, excipients such as, for example, lubricants, plasticisers, anti-tack agents, opacifying agents, pigments, and such like. As will be appreciated by those skilled in the art, the exact choice of excipient and their relative amounts will depend to some extent on the final oral dosage form.

The gastro-resistant and ethanol-resistant compounds disclosed herein are ethanol-insoluble. Accordingly, a skilled artisan will appreciate that the administration of the composition wherein hydromorphone release is controlled by these compounds will not result in increased levels of hydromorphone in the blood of a patient who has or will have consumed an equivalent of as much as 47 ml of alcohol (an equivalent of eight ounces of a 20% ethanol solution, which is a typical alcohol content for a mixed drink) within eight hours of the administration of a composition according to the embodiments of this invention.

According to another aspect of the present invention, the compositions of this invention may comprise active agents other than hydromorphone. The compositions of this invention are especially advantageous for the active agents, which are harmful if their release from the formulation is accelerated due to destruction of the coating.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A controlled-release composition comprising hydromorphone or a pharmaceutically acceptable form thereof in association with a gastro-resistant and ethanol-resistant compound.

2. The controlled-release composition of claim 1, said controlled-release composition being in the form of a capsule, a tablet, a pill, a pellet or a granule.

3. The controlled-release composition of claim 2 wherein the gastro-resistant and ethanol-resistant compound is in a matrix of the composition.

4. The controlled-release composition of claim 3, further comprising at least one encapsulating agent.

5. The controlled-release composition of claim 2, wherein the gastro-resistant and ethanol-resistant compound comprises a coating of the controlled-release composition.

6. The controlled-release composition of claim 5, further comprising at least one encapsulating agent, wherein said at least one encapsulating agent forms the outermost layer of the controlled release composition.

7. The controlled-release composition of claim 5, further comprising at least one encapsulating agent, wherein said at least one encapsulating agent forms a layer located between a matrix and a coating of the controlled-release composition.

8. The controlled-release composition of claim 2, wherein the gastro-resistant and ethanol-resistant compound is selected from the group consisting of alginic acid, glyceryl monostearate, glyceryl palmitostearate, carnauba wax, microcrystalline wax, white wax, yellow wax, ethylcellulose with less than 46.5% of ethonyl groups, and any combination thereof.

9. A method of treating a mammal having chronic pain comprising orally administering such mammal the composition of claim 2 once a day, wherein administration of said composition to the mammal who has or will have consumed an equivalent of 47 ml of ethanol within 8 hours of administration does not result in an elevated level of hydromorphone in blood of said mammal.

10. The method of claim 9, wherein said mammal is a human.

11. A controlled-release composition comprising one or more active agents in association with a gastro-resistant and ethanol-resistant compound, wherein an accelerated release of at least one of said one or more active agents from said controlled-release composition is potentially harmful to a subject who intakes said controlled-release composition.
